(19) United States
(12) Patent Application Publication
(10) Pub. No.: US 2023/0241023 A1
(43) Pub. Date: Aug. 3, 2023

(54) STABILIZATION OF 1,2,4-TRIOXANE COMPOUNDS BY CHLOROGENIC ACIDS

(71) Applicants: Artemiflow GmbH, Potsdam (DE); Max-Planck-Gesellschaft zur Forderung Der Wissenschaften e.V., Munich (DE)

(72) Inventors: Adam Jeffrey MAUST, Dauphin, PA (US); Kerry GILMORE, Ashford, CT (US)

(21) Appl. No.: 18/007,990

(22) PCT Filed: Jun. 2, 2021
(86) PCT No.: PCT/EP2021/064859

§ 371 Date: Dec. 2, 2022

Related U.S. Application Data
(60) Provisional application No. 63/033,509, filed on Jun. 2, 2020.

(51) Int. Cl. A61K 31/357; A61K 47/14; A61K 36/82; A61K 36/74; A23L 33/105; A23L 2/52; A23K 10/30; A23K 20/111; A23K 20/121; A23K 20/126

(52) U.S. Cl. CPC A61K 31/357; A61K 47/14; A61K 36/82; A61K 36/74; A23L 33/105; A23L 2/52; A23K 10/30; A23K 20/111; A23K 20/121; A23K 20/126; A61K 2236/331; A61K 2236/37

(57) ABSTRACT

The present invention relates to compositions comprising 1,2,4-trioxane compounds and chlorogenic acids. The invention further provides a method for the stabilization of 1,2,4-trioxane compounds against degradation, in particular against thermal degradation or degradation induced by reducing agents such as reducing carbohydrates, reducing sugars, electromagnetic radiation or heavy metal ions.

### FIELD OF THE INVENTION

The present invention relates to compositions comprising 1,2,4-trioxane compounds and chlorogenic acids. The invention further provides a method for the stabilization of 1,2,4-trioxane compounds against degradation, in particular against thermal degradation or degradation induced by reducing agents such as reducing carbohydrates, reducing sugars, electromagnetic radiation or heavy metal ions.

### BACKGROUND

1,2,4-Trioxane compounds, in particular Artemisinin and its natural, semisynthetic and synthetic derivatives have gained significant attention due to their broad spectrum of health benefits. Besides the well known application in antimalarials, Li et al., Antiviral Research 67, (2005), p. 18-23 reportated that extracts of sweet wormwood (Artemisia annua) exhibit antiviral activity against SARS-CoV.

A review of Efferth, Biology Advances 36 (2018), 1730-1737 on antiviral activity of artemisinin type compounds draws the conclusion that Artemisia annua extracts containing artemisinin and other compounds seem to be active against double stranded DNA viruses but less active towards other viruses and in particular leaves the question open whether such extracts are effective against single stranded RNA viruses.

Several authors have demonstrated the potential of artemisinin and its derivatives to be repurposed for use in anti-cancer regimens (see Tsuda, K. et al. Mechanisms of the pH- and oxygen-dependent oxidation activities of artesunate. Biol Pharm Bull 41, 555-563 (2018); Wang, B., Hou, D., Liu, Q., Wu, T., Guo, H., Zhang, X., Zou, Y., Liu, Z., Liu, J., Wei, J., Gong Y. & Shao, C. Artesunate sensitizes ovarian cancer cells to cisplatin by downregulating RAD51. Cancer Biology & Therapy 16, 1548-1556 (2015); Chen, X., Wong, Y. K., Lim, T. K., Lim, W. H., Lin, Q., Wang, J. & Hua, Z. Artesunate activates the intrinsic apoptosis of HCT116 cells through the suppression of fatty acid synthesis and the NF-κB pathway. Molecules 22, 1272 (2017); Kumar, B., Kalvala, A., Chu, S., Rosen, S., Forman, S. J., Marcucci, G., Chen, C. C. & Pullarkat, V. Antileukemic activity and cellular effects of the antimalarial agent artesunate in acute myeloid leukemia. Leuk Res 59, 124-135 (2017); Liu, Y., Gao, S., Zhu, J., Zheng, Y., Zhang, H, & Sun H. Dihydroartemisinin induces apoptosis and inhibits proliferation, migration, and invasion in epithelial ovarian cancer via inhibition of the hedgehog signaling pathway. Cancer Med 7, 5704-5715 (2018); Greenshields, A., Shepherd, T. & Hoskin, D. Contribution of reactive oxygen species to ovarian cancer cell growth arrest and killing by the anti-malarial drug artesunate. Molecular Carcinogenesis 56, 75-93 (2017).

However, 1,2,4-trioxane such as artemisinin being peroxides are prone to degradation upon thermal stress, in the presence of heavy metal ions, reducing agents and upon exposure to electromagnetic radiation which limits their use and precessability.

Surprisingly, combinations were found which stabilize 1,2,4-trioxanes significantly.

### SUMMARY OF THE INVENTION

The present invention relates to compositions comprising

a) at least one compound having at least one 1,2,4-trioxane moiety and

b) at least one chlorogenic acid.

It was found that these compositions increase stability of compounds having at least one 1,2,4-trioxane moiety in particular when exposed to reducing sugars, thermal stress, light or other electromagnetic radiation and heavy metal ions and thus are useful e.g. in medical and veterinary applications, in foods and beverages, as food and animal feed additives and as nutraceuticals.

The invention further provides medical and veterinary compositions, foods and beverages, food and animal feed additives and nutraceuticals comprising the aforementioned compositions as well as the use of chlorogenic acids for the stabilization of compounds having at least one 1,2,4-trioxane moiety against degradation.

### DETAILED DESCRIPTION

For purposes of interpreting this specification, the following definitions will apply, and whenever appropriate, terms used in the singular will also include the plural.

Terms used in the specification have the following meanings unless the context clearly indicates otherwise:

As used herein, the term “a,” “an,” “the” and similar terms used in the context of the present invention especially in the context of the claims are to be construed to cover both the singular and plural unless otherwise indicated herein or explicitly contradicted by the context.

All methods described herein can be performed in any suitable order unless otherwise indicated herein or otherwise clearly contradicted by context. The use of any and all examples, or exemplary language e.g. “such as” provided herein is intended merely to better illustrate the invention and does not pose a limitation on the scope of the invention otherwise claimed.

“Optionally substituted” or “substituted” means one or more hydrogen atoms at any position in the molecule or moiety referred to can be substituted by any one or any combination of substituents with their number, placement and selection being understood to encompass only those substitutions that a skilled chemist would expect to be reasonably stable.

Various embodiments of the invention are described herein. It will be recognized that features specified in each embodiment may be combined with other specified features to provide further embodiments.

The compositions according to the invention comprise at least one compound comprising at least one 1,2,4-trioxane moiety, such compounds being those comprising at least one 1,2,4-trioxane ring which is optionally, but preferably substituted.

In one embodiment the compounds comprising at least one 1,2,4-trioxane moiety are selected from those of formulae (I) to (V)

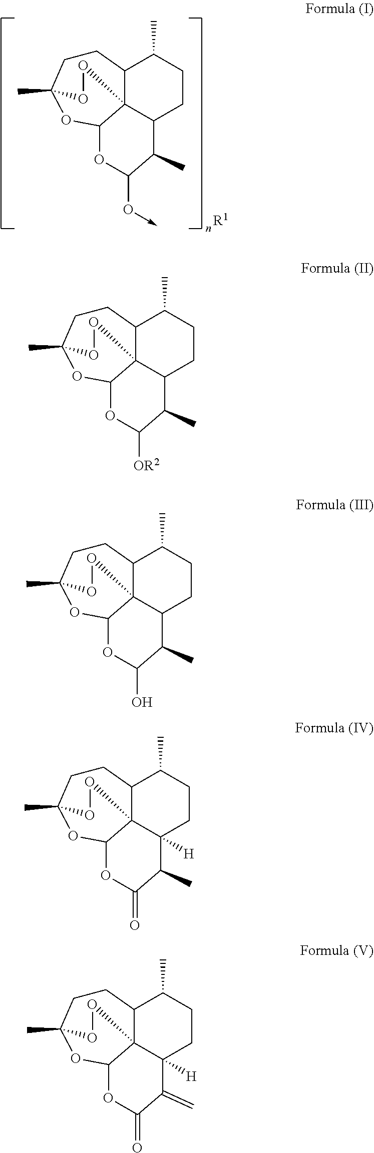

and, where applicable, pharmaceutically acceptable salts of the aforementioned compounds of formulae (I) and (II).

In formula (I)

the arrow denotes the bond between the depicted oxygen atom to the residue IV
n is an integer of more than 1, preferably 2 to 10, more preferably 2, 3 or 4 and even more preferably 2 or 3
R¹ is a residue that is n times substituted by the residue depicted in the rounded bracket, and is preferably C₁-C₁₈-alkyl or C₂-C₁₈-alkenyl or —(CO)ₙ(R³), wherein the carboyl groups together with the oxygen bound to the residue R¹ form a carboxylic ester moiety and R³ is C₁-C₁₈-alkane-n-yl or C₂-C₁₈-alkene-n-yl
   whereby
   the aforementioned C₁-C₁₈-alkyl, C₂-C₁₈-alkenyl, C₁-C₁₈-alkane-n-yl, C₂-C₁₈-alkene-n-yl groups are
either not, once, twice, or more than twice interrupted by non-successive functional groups selected from the group consisting of:
   —O—, —S—, —SO₂—, —SO—, —SO₂NR⁴—, NR⁴SO₂—, —NR⁴—, —CO—, —O(CO)—, (CO)O—, —O(CO)O—, —NR⁴(CO)NR⁴—, NR⁴(CO)—, —(CO)NR⁴—, —NR⁴(CO)O—, —O(CO)NR⁴—,
      and
either not, additionally, or alternatively either once, twice or more than twice interrupted by bivalent residues selected from the group consisting of heterocyclo-diyl, and aryldiyl,
   and
either not, additionally, or alternatively either once, twice or more than twice substituted by substituents selected from the group consisting of:
   hydroxy, halogen, cyano, azido, C₆-C₁₄-aryl, C₁-C₈-alkoxy, C₁-C₈-alkylthio, —SO₃M, —COOM, PO₃M₂, —PO(N(R⁵)₂)₂, PO(OR⁵)₂, —SO₂N(R⁴)₂, —N(R⁴)₂, —CO₂N(R⁵)₂, —COR⁴, —OCOR⁴, —NR⁴(CO)R⁵, —(CO)OR⁴, —NR⁴(CO)N(R⁴)₂.

In formula (II)
R² is C₁-C₁₈-alkyl or C₂-C₁₈-alkenyl or —(CO)R³, wherein the carboyl groups together with the oxygen bound to the residue R¹ form a carboxylic ester moiety and R³ is C₁-C₁₈-alkyl or C₂-C₁₈-alkenyl whereby
   the aforementioned C₁-C₁₈-alkyl and C₂-C₁₈-alkenyl groups are
      either not, once, twice, or more than twice interrupted by non-successive functional groups selected from the group consisting of:
         —O—, —S—, —SO₂—, —SO—, —SO₂NR⁴—, NR⁴SO₂—, —NR⁴—, —CO—, —O(CO)—, (CO)O—, —O(CO)O—, —NR⁴(CO)NR⁴—, NR⁴(CO)—, —(CO)NR⁴—, —NR⁴(CO)O— or —O(CO)NR⁴—
      and
      either not, additionally, or alternatively either once, twice or more than twice interrupted by bivalent residues selected from the group consisting of heterocyclo-diyl, and aryldiyl,
      and
      either not, additionally, or alternatively either once, twice or more than twice substituted by substituents selected from the group consisting of:
         hydroxy, halogen, cyano, azido, C₆-C₁₄-aryl, C₁-C₈-alkoxy, C₁-C₈-alkylthio, —SO₃M, —COOM, PO₃M₂, —PO(N(R⁵)₂)₂, PO(OR⁵)₂, —SO₂N(R⁴)₂, —N(R⁴)₂, —CO₂N(R⁵)₂, —COR⁴, —OCOR⁴, —NR⁴(CO)R⁵, —(CO)OR⁴ or —NR⁴(CO)N(R⁴)₂
            whereby in all formulae above where used
R⁴ is independently selected from the group consisting of hydrogen, C₁-C₈-alkyl, C₆-C₁₄-aryl, and heterocyclyl or N(R⁴)₂ as a whole is a N-containing heterocycle,
R⁵ is independently selected from the group consisting of C₁-C₈-alkyl, C₆-C₁₄-aryl, and heterocyclyl or N(R⁵)₂ as a whole is a N-containing heterocycle and
M is hydrogen, or 1/q equivalent of an q-valent metal ion or is an ammonium ion or a guanidinium ion or a primary, secondary, tertiary or quarternary organic ammonium ion, in particular those of formula [N(C₁-C₁₈-alkyl)ₛHₜ]⁺ wherein s is 1,2,3 or 4 and t is (4-s).

As used herein, and unless specifically stated otherwise, C₁-C₁₈-alkyl, C₁-C₁₈-alkene-n-yl, C₁-C₈-alkyl, C₁-C₈-alkoxy and C₁-C₈-alkylthio include straight-chained or, for C₃-C₁₈ or C₃-C₈ also cyclic either in part or as a whole, branched or unbranched alkyl, alkoxy, and alkylthio substituents having the given number of carbon atoms in the substituent as such.

As used herein, and unless specifically stated otherwise, C₂-C₁₈-alkenyl include straight-chained or, for C₅-C₁₈ also cyclic either in part or as a whole, branched or unbranched alkenyl, having the given number of carbon atoms in the substituent as such.

As used herein, and unless specifically stated otherwise, C₆-C₁₄-aryl, C₆-C₁₄-aryloxy, and C₆-C₁₄-arylthio denote carbocyclic aromatic substituents having six to fourteen carbon atoms within the aromatic system as such, i.e. without carbon atoms of substituents, preferably phenyl (C₆), naphthyl (C₁₀), phenanthrenyl and anthracenyl (each C₁₄), whereby said carbocyclic, aromatic substituents are either unsubstituted or substituted by up to five identical or different substituents per cycle. For example and with preference, the substituents are selected from the group consisting of fluoro, chloro, C₁-C₁₈-alkyl, C₁-C₁₈-alkoxy, C₆-C₁₄-aryl.

In a more preferred embodiment the carbocyclic, aromatic substituents are unsubstituted.

Specific examples of C₁-C₁₈-alkyl are methyl, ethyl, n-propyl, isopropyl, n-butyl, sec-butyl, tert-butyl, n-pentyl, isopentyl, tert.-pentyl, neopentyl, cyclohexyl, n-hexyl, n-heptyl, n-octyl and isooctyl, n-decyl, n-dodecyl n-hexadecyl, n-octadecyl.

Specific examples of C₁-C₈-alkoxy-substituents are methoxy, ethoxy, isopropoxy, n-propoxy, n-butoxy, sec.-butoxy, tert-butoxy and cyclohexyloxy.

Specific examples of C₁-C₈-alkylthio-substituents are methylthio and ethylthio.

Specific examples of C₆-C₁₄-aryl are phenyl, o-, m-and p-tolyl.

A further specific example of an C₆-C₁₄-aryl-substituent is phenoxy.

A further specific example of an C₆-C₁₄-aryl-substituent is phenylthio.

Preferred compounds of formula (II) are those of formula (IIa), artemether, and of formula (IIb), artesunate, and pharmaceutically acceptable salts of artesunate.

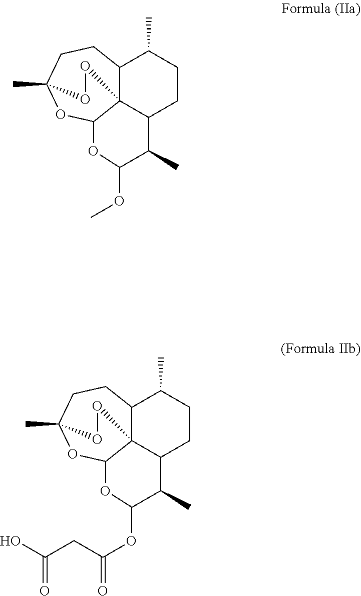

The compound of formula (III) is dihydroartemisin.

The compound of formula (IV) is artemisinin.

The compound of formula (V) is artemisitene.

In one embodiment of the invention the compositions according to the invention contain more than one compound comprising at least one 1,2,4-trioxane moiety and preferably more than one compound selected from those of formulae (I) to (V) above and, where applicable, pharmaceutically acceptable salts of such compounds.

In one preferred embodiment the compositions according to the invention comprises at least two, for example two, three, four or all the compounds selected from formula (IIa), (IIb), (III), (IV) and (V).

As a natural source of compounds comprising at least one 1,2,4-trioxane moiety the plant Artemisia annua or parts thereof as such may be employed or extracts obtained via known methods from Artemisia annua as and, where desired, standard workup methods e.g. as published in Triemer et al., Angewandte Chemie, International Edition 57, (2018), p. 5525-5528.

Where Artemisia annua is extracted, this may occur using the whole plant or parts thereof such as leaves or stems, whether dried or freshly harvested. Suitable solvents for extraction include hexanes, cyclohexane, supercritical carbon dioxide, hydrofluorocarbon HFC-134a, ionic liquids, water, methanol, ethanol, 1-butanol, acetone, cyclohexanone, toluene, ethyl acetate, acetonitrile, tetrahydrofuran, or mixtures thereof.

Where e.g. single compounds comprising at least one 1,2,4-trioxane moiety of the invention are desired to be used in the composition extracts of Artemisia annua can be separated in a manner known per se to obtain the individual compounds for example, by partitioning between polyphasic solvent mixtures, recrystallization and/or chromatographic separation, for example over silica gel or by, e.g., medium pressure liquid chromatography over a reversed phase column or by fractional crystallization.

In one embodiment the Artemisia annua plant is of the Apollon variety, see X. Simmonet et al., “Apollon, a new Artemisia annua variety with high artemisinin content”, Planta Medica, 2011, 77(12) which is commercially available from the company Mediplant, Conthey Switzerland.

The individual compounds comprising at least one 1,2,4-trioxane moiety can be worked up and/or purified according to standard methods, e.g., using chromatographic methods, distribution methods, (re-) crystallization, and the like.

Synthetic or semi-synthetic compounds comprising at least one 1,2,4-trioxane moiety are for example prepared by preparation methods known to those skilled in the art and some of which are published e.g. in Reiter, C., Fröhlich, T., Gruber, L., Hutterer, C., Marschall, M., Voigtländer, C., Friedrich, O., Kappes, B., Efferth, T., Tsogoeva, S. B., 2015a. Highly potent artemisinin-derived dimers and trimers: Synthesis and evaluation of their antimalarial, antileukemia and antiviral activities. Bioorg. Med. Chem. 23 (17), 5452-5458; Reiter, C., Fröhlich, T., Zeino, M., Marschall, M., Bahsi, H., Leidenberger, M., Friedrich, O., Kappes, B., Hampel, F., Efferth, T., Tsogoeva, S. B., 2015b. New efficient artemisinin derived agents against human leukemia cells, human cytomegalovirus and Plasmodium falciparum: 2nd generation 1,2,4-trioxane-ferrocene hybrids. Eur. J. Med. Chem. 97, 164-172; Posner, G. H., Ploypradith, P., Parker, M. H., O'Dowd, H., Woo, S. H., Northrop, J., Krasavin, M., Dolan, P., Kensler, T. W., Xie, S., Shapiro, T. A., 1999. Antimalarial, antiproliferative, and antitumor activities of artemisinin-derived, chemically robust, trioxane dimers. J. Med. Chem. 42 (21), 4275-4280; Paik, I. H., Xie, S., Shapiro, T. A., Labonte, T., Narducci Sarjeant, A. A., Baege, A. C., Posner, G. H., 2006. Second generation, orally active, antimalarial, artemisinin-derived trioxane dimers with high stability, efficacy, and anticancer activity. J. Med. Chem. 49 (9), 2731-2734, Li, Y., Zhu, Y. M., Jiang, H. J., Pan, J. P., Wu, G. S., Wu, J. M., Shi, Y. L., Yang, J. D., Wu, B. A., 2000. Synthesis and antimalarial activity of artemisinin derivatives containing an amino group. J. Med. Chem. 43 (8), 1635-1640; Ren, Y., Yu, J., Kinghorn, A. D., 2016. Development of anticancer agents from plant-derived sesquiterpene lactones. Curr. Med. Chem. 23 (23), 2397-2420. O'Neill, P. M., Searle, N. L., Kan, K. W., Storr, R. C., Maggs, J. L., Ward, S. A., Raynes, K., Park, B. K., 1999. Novel, potent, semisynthetic antimalarial carba analogues of the first-generation 1,2,4-trioxane artemether. J. Med. Chem. 42 (26), 5487-5493 which are hereby incorporated by reference.

The compositions according to the invention further comprise at least one chlorogenic acid.

As used herein the term “chlorogenic acid” or chlorogenic acids” denote compounds wherein one or two hydroxyl groups of quinic acid are esterified with caffeic, ferulic or p-coumaric acid.

Preferred examples of chlorogenic acids include 3-O-caffeoylquinic acid (formula VI a), 4-O-caffeoylquinic acid (formula VI b), 5-O-caffeoylquinic acid (formula VI c), 3-O-ferruoylquinic acid (formula VI d), 4-O-ferruoylquinic acid (formula VI e), 5-O-ferruoylquinic acid (formula VI f), 3,4-dicaffeoylquinic acid (formula VII a), 3,5-dicaffeoylquinic acid (formula VII b) and 4,5-dicaffeoylquinic acid (formula VII c), whereby 3-O-caffeoylquinic acid (formula VI a), 4-O-caffeoylquinic acid (formula VI b), 5-O-caffeoylquinic acid (formula VI c), 3,4-dicaffeoylquinic acid (formula VII a), 3,5-dicaffeoylquinic acid (formula VII b) and 4,5-dicaffeoylquinic acid (formula VII c) are even more preferred.

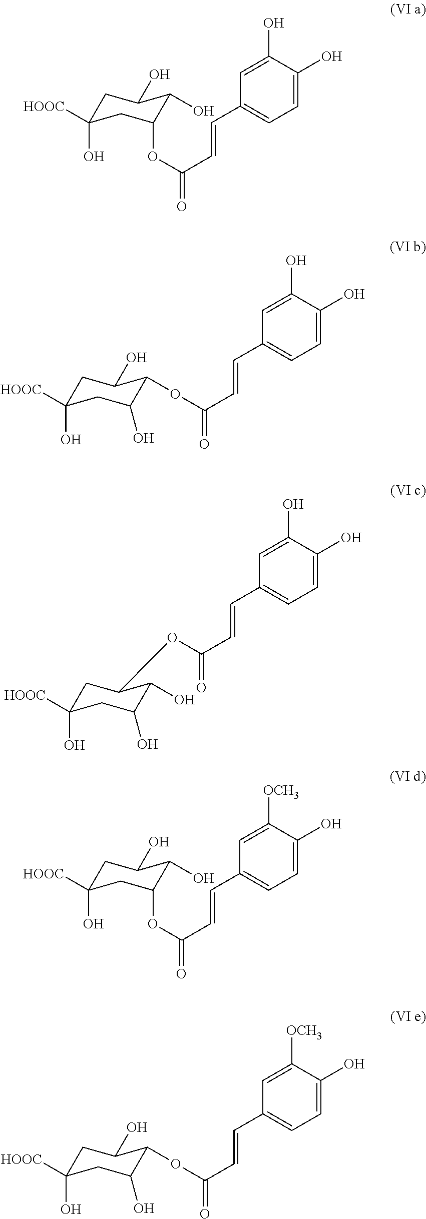 [SMILES: O=C(/C=C/c1ccc(O)c(O)c1)OC1CC(O)(C(=O)O)CC(O)C1O ; O=C(/C=C/c1ccc(O)c(O)c1)OC1C(O)CC(O)(C(=O)O)CC1O ; O=C(/C=C/c1ccc(O)c(O)c1)OC1CC(O)(C(=O)O)CC(O)C1O ; COc1cc(/C=C/C(=O)OC2CC(O)(C(=O)O)CC(O)C2O)ccc1O ; COc1cc(/C=C/C(=O)OC2C(O)CC(O)(C(=O)O)CC2O)ccc1O]

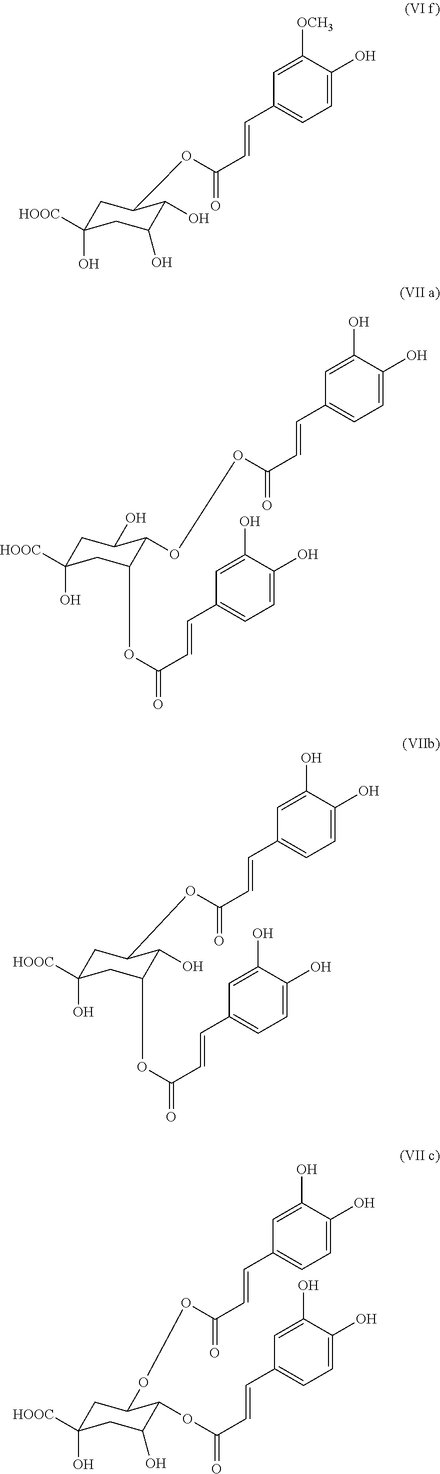 [SMILES: COc1cc(/C=C/C(=O)OC2CC(O)(C(=O)O)CC(O)C2O)ccc1O ; O=C(/C=C/c1ccc(O)c(O)c1)OOC1C(O)CC(O)(C(=O)O)CC1OC(=O)/C=C/c1ccc(O)c(O)c1 ; O=C(/C=C/c1ccc(O)c(O)c1)OC1CC(O)(C(=O)O)CC(OC(=O)/C=C/c2ccc(O)c(O)c2)C1O ; O=C(/C=C/c1ccc(O)c(O)c1)OOC1CC(O)(C(=O)O)CC(O)C1OC(=O)/C=C/c1ccc(O)c(O)c1]

In one embodiment, the molar ratio between the compound or the compounds having at least one 1,2,4-trioxane moiety and the chlorogenic acid or chlorogenic acids present in the composition according to the invention is for example from 3 to 0.001, preferably from 0.7 to 0.003, more preferably from 0.4 to 0.01 and even more preferably from 0.1 to 0.01.

Chlorogenic acids may be used in their isolated form or as component of whole plants, plant parts or extracts of the aformentioned. As for Artemisia annua chlorogenic acids can be separated in a manner known per se to obtain the individual compounds for example, by partitioning between polyphasic solvent mixtures, recrystallization and/or chromatographic separation, for example over silica gel or by, e.g., medium pressure liquid chromatography over a reversed phase column or by fractional crystallization.

The individual chlorogenic acids can be worked up and/or purified according to standard methods, e.g., using chromatographic methods, distribution methods, (re-) crystallization, and the like.

Due to its high content of chlorogenic acid coffee, in particular roasted coffee can be used as a valuable source of chlorogenic acids.

In one embodiment the combinations according to the invention can be obtained by co-extracting coffee, in particular roasted coffee, with Artemisia annua, in particular dried leaves of Artemisia annua, e.g. with water, preferably at a temperature of 40 to 100° C., more preferably 50 to 100° C. The weight ratio of roasted coffee to Artemisia annua is for example from 1 to 50, preferably from 5 to 50. Coffees include those of the variety robusta (Coffea canephora) and arabica (Coffea arabica).

Suitable extracts also encompass teas comprising Artemisia annua such as black teas, teas comprising cinnamon and/or licorice.

The invention further comprises beverages, foods and animal feeds, nutraceuticals and food or animal feed additives comprising the composition of any of the preceding embodiments, in particular those further comprising reducing sugars or carbohydrates.

As used herein reducing sugars include galactose, glucose, glyceraldehyde, fructose, ribose, and xylose, lactose, maltose and sucrose. Reducing carbohydrates include starch, maltodextrine and glycogen.

Beverages include teas, infusions, coffee and coffee beverages, beers, wines, sparkling wines, milk, lemonades, alcoholic beverages, soft drinks and fruit juices.

The compounds of formulae (VIII) to (XVIII) below were reported to be present in Artemisia annua extracts, see inter alia Czechowski et al., Frontiers in Plant Science, 2019, Vol. 10, Article 984; Zarelli et al., Phytochemical Analysis 2019, 30, 564-571.

Therefore, the compositions according to the invention may further include at least one compound, for example one, two, three, four, five, six, seven, eight, nine, ten or all compounds selected from the group consisting of those of formulae (VIII) to (XVIII)

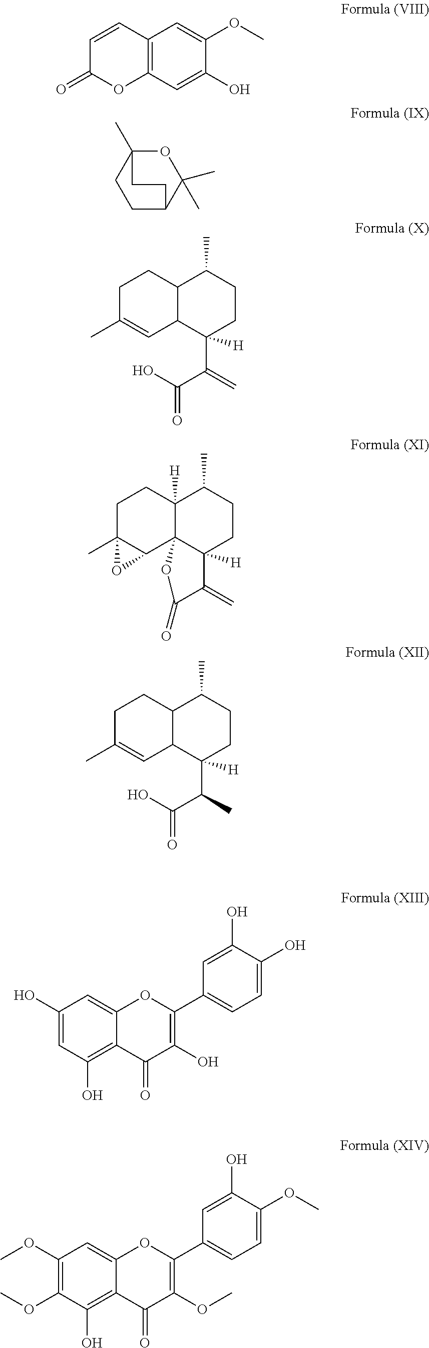

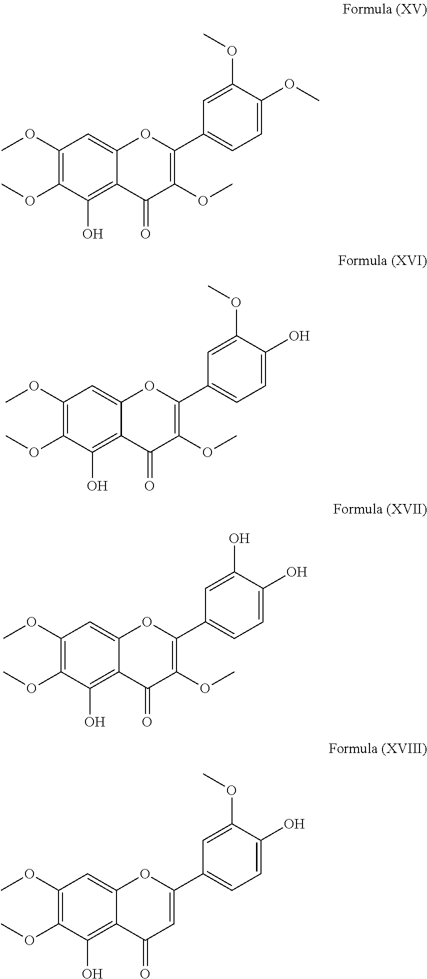

The compound of formula (VIII) is scopoletin.

The compound of formula (IX) is 1,8-cineole.

The compound of formula (X) is artemisinic acid.

The compound of formula (XI) is arteannuin-B.

The compound of formula (XII) is dihydroartemisinic acid.

The compound of formula (XIII) is fisetin.

The compound of formula (XIV) is casticin.

The compound of formula (XIV) is artemetin.

The compound of formula (XVI) is chrysoplenetin.

The compound of formula (XVII) is chrysoplenol-D.

The compound of formula (XVIII) is cirsilineol.

The advantage of the invention is that by combination with chlorogenic acids and thus stabilization of 1,2,4-trioxane compounds against degradation their scope of application and processability is significantly broadened and thus allows for these compounds to be used in applications that were not known before.

The invention is further exemplified hereinbelow without, however, limiting the scope of the invention.

### Experimental

### Reagents and Materials

Solvents were obtained from commercial suppliers and used without further purification. Dried leaves of Artemisia annua were obtained from ArtemiLife Corp. and Artemisinin was previously prepared and purified by crystallization using the protocols detailed in Horváth, Z.; Horosanskaia, E.; Lee, J. W.; Lorenz, H.; Gilmore, K.; Seeberger, P. H.; Seidel-Morgenstern, A. Recovery of Artemisinin from a Complex Reaction Mixture Using Continuous Chromatography and Crystallization. Org. Process Res. Dev. 2015, 19, 624-634. The Artemisinin crystals obtained thereby were ground prior to use.

Ground coffee used in extractions was preground 100% Arabica beans from Café Intención. Coffee was prepared in a Rowenta C T 3818 Milano coffee maker using size 4 coffee filters (Melitta brand). Coffee beans (100% Arabica) were preground from. 100 g of ground coffee was added to the cone coffee filter in the machine and 1350 mL of water used to make the coffee using the single setting. Coffee was then transferred to a thermos, stored at room temperature, and used without further modification.

### Stability of Artemisinin tests

### Stability of Artemisinin in Presence of Additives

The stability of artemisinin was tested in in aqueous solutions and in the presence of reductants and traces of heavy metal ions (here iron ions) from tap water. Two solvents were tested, tap water and premade black coffee. As a reductant glucose (1 weight equivalent) was tested. All mixtures were heated to 93° C. for 90 minutes in 10 mL glass vials open to the atmosphere. Halfway through heating, 1-2 mL of tap water was added to compensate for evaporation. Following heating, 2 mL ethyl acetate was added, and the biphasic solution was dried using a rotary evaporator with water bath at 40° C. The flask was further dried for three hours under high vacuum. All samples were dissolved in CDCl₃ containing 0.5 equivalents of an internal standard (1,2,4,5-tetramethylbenzene) and ¹H NMR performed.

### Example 1 (For Comparison)

30.8 mg of ground artemisinin and 18.2 mg glucose (1 mol equivalent) were added to 5 mL of tap water. The mixture was heated for 90 minutes prior to cooling, ethyl acetate addition, and evaporation. 64.1 mg of a yellowish oil/plaque with white spots was obtained. ¹H NMR revealed 32% clean loss of artemisinin. No degradation products were identified.

### Example 2 (Inventive)

30.5 mg of ground artemisinin and 19.6 mg glucose (1 mol equivalent) were added to 5 mL of premade black coffee. The mixture was heated for 90 minutes prior to cooling, ethyl acetate addition, and evaporation. 302 mg of a brown/black solid was obtained. ¹H NMR revealed 19% clean loss of artemisinin. No degradation products were identified.

Compared to example 1 much less artemisin was degraded showing the stabilization effect of chlorogenic acids.

1. A composition comprising
a) at least one compound having at least one 1,2,4-trioxane moiety and
b) at least one chlorogenic acid.

2. The composition according to claim 1, wherein the compounds comprising at least one 1,2,4-trioxane moiety are selected from those of formulae (I) to (V) 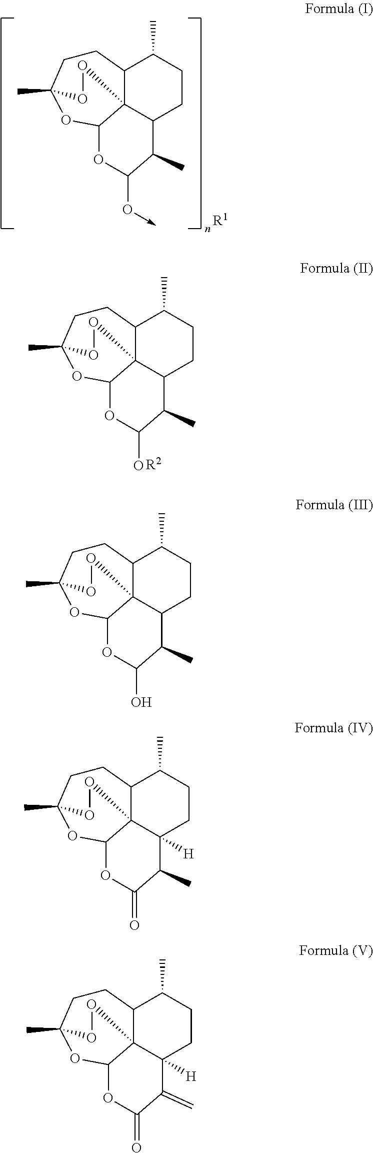
and, where applicable, pharmaceutically acceptable salts of the aforementioned compounds of formulae (I) and (II) wherein
in formula (I)
the arrow denotes the bond between the depicted oxygen atom to the residue R¹
n is an integer of more than 1, preferably 2 to 10, more preferably 2, 3 or 4 and even more preferably 2 or 3
R¹ is a residue that is n times substituted by the residue depicted in the rounded bracket, and is preferably C₁-C₁₈-alkyl or C₂-C₁₈-alkenyl or —(CO)ₙ(R³), wherein the carboyl groups together with the oxygen bound to the residue R¹ form a carboxylic ester moiety and R³ is C₁-C₁₈-alkane-n-yl or C₂-C₁₈-alkene-n-yl
whereby
the aforementioned C₁-C₁₈-alkyl, C₂-C₁₈-alkenyl, C₁-C₁₈-alkane-n-yl, C₂-C₁₈-alkene-n-yl groups are
either not, once, twice, or more than twice interrupted by non-successive functional groups selected from the group consisting of:
—O—, —S—, —SO₂—, —SO—, —SO₂NR⁴—, NR⁴SO₂—, —NR⁴—, —CO—, —O(CO)—, (CO)O—, —O(CO)O—, —NR⁴(CO)NR⁴—, NR⁴(CO)—, —(CO)NR⁴—, —NR⁴(CO)O—, —O(CO)NR⁴—,
and
either not, additionally, or alternatively either once, twice or more than twice interrupted by bivalent residues selected from the group consisting of heterocyclo-diyl, and aryldiyl,
and
either not, additionally, or alternatively either once, twice or more than twice substituted by substituents selected from the group consisting of:
hydroxy, halogen, cyano, azido, C₆-C₁₄-aryl, C₁-C₈-alkoxy, C₁-C₈-alkylthio, —SO₃M, —COOM, PO₃M₂, —PO(N(R⁵)₂)₂, PO(OR⁵)₂, —SO₂N(R⁴)₂, —N(R⁴)₂, —CO₂N(R⁵)₂, —COR⁴, —OCOR⁴, —NR⁴(CO)R⁵, —(CO)OR⁴, —NR⁴(CO)N(R⁴)₂.
and in formula (II)
R² is C₁-C₁₈-alkyl or C₂-C₁₈-alkenyl or —(CO)R³, wherein the carboyl groups together with the oxygen bound to the residue R¹ form a carboxylic ester moiety and R³ is C₁-C₁₈-alkyl or C₂-C₁₈-alkenyl
whereby
the aforementioned C₁-C₁₈-alkyl and C₂-C₁₈-alkenyl groups are
either not, once, twice, or more than twice interrupted by non-successive functional groups selected from the group consisting of:
—O—, —S—, —SO₂—, —SO—, —SO₂NR⁴—, NR⁴SO₂—, —NR⁴—, —CO—, —O(CO)—, (CO)O—, —O(CO)O—, —NR⁴(CO)NR⁴—, NR⁴(CO)—, —(CO)NR⁴—, —NR⁴(CO)O— or —O(CO)NR⁴—
and
either not, additionally, or alternatively either once, twice or more than twice interrupted by bivalent residues selected from the group consisting of heterocyclodiyl, and aryldiyl,
and
either not, additionally, or alternatively either once, twice or more than twice substituted by substituents selected from the group consisting of:
hydroxy, halogen, cyano, azido, C₆-C₁₄-aryl, C₁-C₈-alkoxy, C₁-C₈-alkylthio, ⁻SO₃M, —COOM, PO₃M₂, —PO(N(R⁵)₂)₂, PO(OR⁵)₂, —SO₂N(R⁴)₂, —N(R⁴)₂, —CO₂N(R⁵)₂, —COR⁴, —OCOR⁴, —NR⁴(CO)R⁵, —(CO)OR⁴ or —NR⁴(CO)N(R⁴)₂
whereby in all formulae above where used
R⁴ is independently selected from the group consisting of hydrogen, C₁-C₈-alkyl, C₆-C₁₄-aryl, and heterocyclyl or N(R⁴)₂ as a whole is a N-containing heterocycle,
R⁵ is independently selected from the group consisting of C₁-C₈-alkyl, C₆-C₁₄-aryl, and heterocyclyl or N(R⁵)₂ as a whole is a N-containing heterocycle and
M is hydrogen, or 1/q equivalent of an q-valent metal ion or is an ammonium ion or a guanidinium ion or a primary, secondary, tertiary or quarternary organic ammonium ion, in particular those of formula [N(C₁-C₁₈-alkyl)ₛHₜ]⁺ wherein s is 1,2,3 or 4 and t is (4-s).

3. The composition according to claim 1 wherein the compounds comprising at least one 1,2,4-trioxane moiety are selected from those of formula (IIa), artemether, and of formula (IIb), artesunate, and pharmaceutically acceptable salts of artesunate.

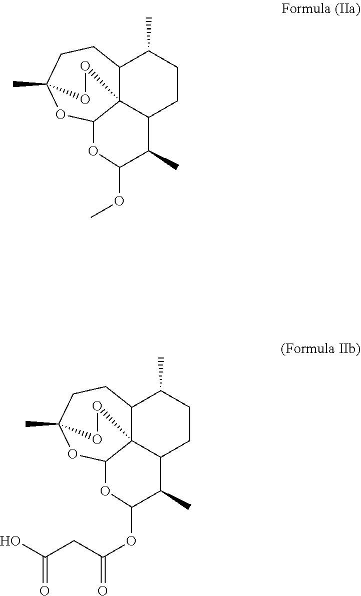

4. The composition according to claim 1, wherein the at least one chlorogenic acid is selected from 3-O-caffeoylquinic acid, 4-O-caffeoylquinic acid, 5-O-caffeoylquinic acid, 3-O-ferruoylquinic acid, 4-O-ferruoylquinic acid, 5-O-ferruoylquinic acid, 3,4-dicaffeoylquinic acid, 3,5-dicaffeoylquinic acid and 4,5-dicaffeoylquinic acid.

5. The composition according to claim 1, wherein the molar ratio between the compound or the compounds having at least one 1,2,4-trioxane moiety and the chlorogenic acid or chlorogenic acids present in the combination is between 2 and 0.002, preferably between 0.8 and 0.005, more preferably between 0.5 and 0.01 and even more preferably 0.1 and 0.01.

6. The composition according to claim 1, wherein the compounds comprising at least one 1,2,4-trioxane moiety are obtained via extraction of Artemisia annua.

7. The composition according to claim 1, wherein the at least one chlorogenic acid is obtained via extraction of coffee or tea.

8. The composition according to claim 1, being obtained via co-extraction of Artemisia annua and coffee and/or tea with water, preferably at 40° C. to 100° C.

9. Medical or veterinary composition comprising a composition according to claim 1.

10. Beverage, food, animal feed, food additive, animal feed additive or nutraceutical comprising a composition according to claim 1.

11. The beverage, food, animal feed, food additive, animal feed additive or nutraceutical according to claim 10 further comprising reducing carbohydrates or reducing sugars.

12. A method for the stabilization of 1,2,4-trioxane compounds against degradation, in particular against thermal degradation or degradation induced by reducing agents such as reducing carbohydrates or reducing sugars or heavy metal ions by adding at least one chlorogenic acid to the 1,2,4-trioxane compound to be stabilized.
